(19) United States
(12) Patent Application Publication
(10) Pub. No.: US 2026/0108653 A1
(43) Pub. Date: Apr. 23, 2026

(54) Volatile Composition Dispenser

(71) Applicant: The Procter & Gamble Company, Cincinnati, OH (US)

(72) Inventors: Stephan Gary BUSH, Liberty Township, OH (US); Faiz Feisal SHERMAN, Mason, OH (US); Pei LI, Eschborn (DE)

(21) Appl. No.: 19/341,260

(22) Filed: Sep. 26, 2025

Related U.S. Application Data
(60) Provisional application No. 63/709,071, filed on Oct. 18, 2024.

(51) Int. Cl. A61L 9/03

(52) U.S. Cl. CPC A61L 9/037; A61L 9/032; A61L 2209/11; A61L 2209/12

(57) ABSTRACT

A volatile composition dispenser includes a reservoir including a volatile composition; an evaporative surface in fluid communication with the reservoir; and an electromagnetic energy source. The evaporative surface includes additives capable of converting electromagnetic energy into thermal energy. A replaceable volatile composition container includes a reservoir comprising a volatile composition; and an evaporative surface in fluid communication with the reservoir. The evaporative surface includes additives capable of converting electromagnetic energy into thermal energy.

### TECHNICAL FIELD

The present disclosure relates to volatile composition dispensers.

### BACKGROUND

Volatile composition dispensers exist for delivering various volatile compositions, such as freshening compositions, into the air. Such volatile composition dispensers may, for example, take the form of a wick-based electrical dispenser having one or more heaters to assist with volatizing the volatile composition into the air. Consumers desire a volatile composition dispenser that utilizes less energy, provides higher performance and/or is a more energy efficient volatile composition dispenser than traditional diffusing devices. This may enable an improved device to function for a substantial period of time using a battery as a power source, rather than using a mains power connection. Therefore, a more energy efficient means of perfume diffusion is needed. This may extend the useful operating life of the device.

### SUMMARY

According to an example of the present disclosure, a volatile composition dispenser includes a reservoir including a volatile composition; an evaporative surface in fluid communication with the reservoir; and an electromagnetic energy source. The evaporative surface includes additives capable of converting electromagnetic energy into thermal energy.

According to another example of the present disclosure, a replaceable volatile composition container includes a reservoir comprising a volatile composition; and an evaporative surface in fluid communication with the reservoir. The evaporative surface includes additives capable of converting electromagnetic energy into thermal energy.

### BRIEF DESCRIPTION OF THE DRAWINGS

The following detailed description of specific examples of the present disclosure can be best understood when read in conjunction with the following drawings, where like structure is indicated with like reference numerals and wherein:

FIG. 1 is a perspective view of volatile composition dispenser, in accordance with examples of the present disclosure.

FIG. 2 is a top-down view of a volatile composition dispenser, in accordance with examples of the present disclosure.

FIG. 3 is a cut-away view of a volatile composition dispenser, in accordance with examples of the present disclosure.

FIG. 4 is a cut-away view of a volatile composition dispenser, in accordance with examples of the present disclosure.

FIG. 5 is a perspective view of a replaceable volatile composition container, in accordance with examples of the present disclosure.

FIG. 6 is a view of how reflection converges on the focal points of an elliptical shape, in accordance with examples of the present disclosure.

FIG. 7 is a view of how reflection converges on the focal points of an elliptical shape, in accordance with examples of the present disclosure.

FIG. 8 is a perspective view of volatile composition dispenser, in accordance with examples of the present disclosure.

FIG. 9 is a perspective view of volatile composition dispenser, in accordance with examples of the present disclosure.

FIG. 10 is a perspective view of volatile composition dispenser, in accordance with examples of the present disclosure.

FIG. 11 is a perspective view of volatile composition dispenser, in accordance with examples of the present disclosure.

FIG. 12 shows a volatile composition dispenser of the present disclosure.

### DETAILED DESCRIPTION

The volatile composition dispenser and/or replaceable volatile composition container of the present disclosure provides a means of evaporating a volatile composition using less energy than the means used in traditional diffusing devices. This may enable an improved device to function for a substantial period of time using a battery as a power source, rather than using a mains power connection. A portable device would ideally be capable of operating from a battery, away from a mains connection, so that it can perform its function of diffusion of perfume during, for example, a special occasion or social function. Ideally this is at least four hours, and preferably 12-24 hours. A typical mains-connected perfume diffusing device may consume approximately 2 Watts. As an example, such a device may be configured with a lithium ion battery having a capacity of 1300 mA-hr, and an average voltage during its discharge of 3.7V, thus enabling it to deliver approximately 4.8 Watt-hours of energy. This device would operate for approximately 2.4 hours. Therefore, a more energy efficient means of perfume diffusion is needed to extend the useful operating life of the device.

The means of perfume volatilization of many diffusing devices is an annulus-shaped heater that surrounds a porous member saturated with perfume liquid. In such an arrangement, the heater must first consume electric energy to increase the temperature of the heater itself. Then, the heater increases the temperature of the air between the heater surfaces and the porous member. Finally, the air increases the temperature of the porous member, the exposed surface of which then releases perfume vapor as the liquid is evaporated. In this arrangement, heat is also lost to air which flows through the device due to buoyant effects and to conduction of heat to adjacent housing members, i.e. portions of the dispenser that are not the evaporative surface. The useful work is to evaporate liquid perfume, which requires only a small fraction of the total energy supplied. Most of the energy is lost to the environment without doing useful work.

A further objective of the examples of the disclosure is to decrease the time required for the perfume diffusing device to evaporate perfume in sufficient quantity such that the user notices the presence of the perfume in the room. In the above-described annular heater arrangement, a large mass of heater, air, and housing components must be heated before volatilization can begin. If the heating of other components can be eliminated, and the evaporating surface heated directly, perfume diffusion into the room can proceed much more quickly.

A further objective of the examples of the disclosure is to increase the maximum rate at which perfume can be delivered from the diffusing device. In the above-described annular heater arrangement, the heater must achieve a much higher temperature than the evaporating surface of the porous member. This is required to drive heat through the insulating layer of air between the heater and porous member, and to overcome the losses to the environment. The heater temperature may be a limiting factor in the maximum delivery rate of the diffusing device, due to the fact that temperatures in the device may be constrained by safety and material compatibility considerations.

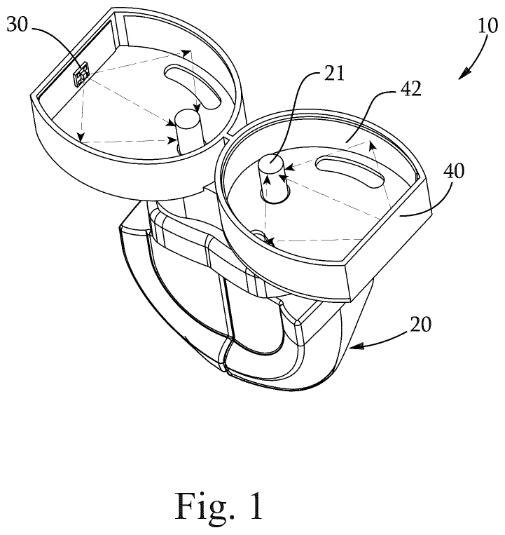

Referring initially to FIGS. 1 and 3, a volatile composition dispenser 10 may include a reservoir 28 including a volatile composition 29, an evaporative surface 21 in fluid communication with the reservoir 28, and an electromagnetic energy source 30. The reservoir 28 may be positioned within a replaceable volatile composition container 20.

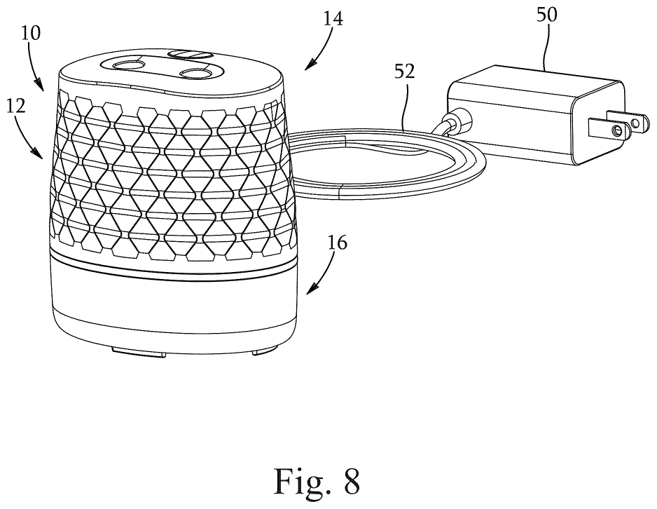

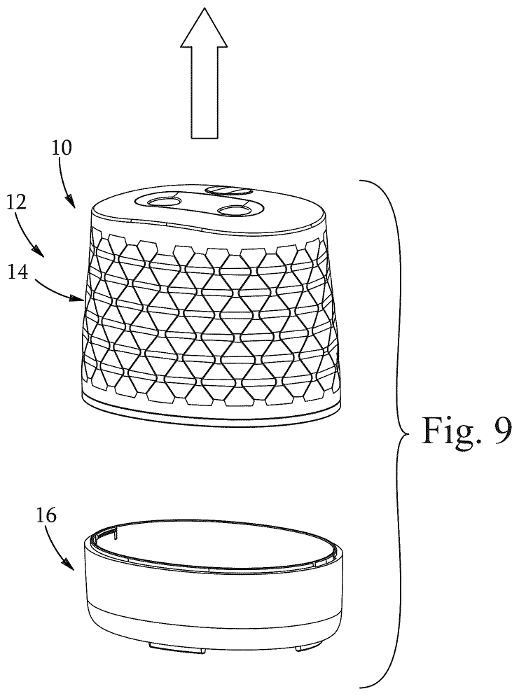

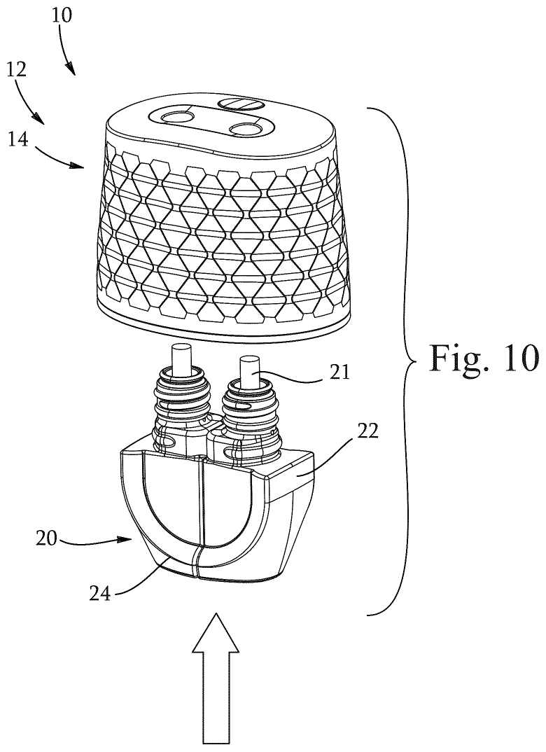

The volatile composition dispenser 10 may include a housing 12 (as shown in FIGS. 8-12) and a replaceable volatile composition container 20. The replaceable volatile composition container 20 may include a reservoir 28 comprising a volatile composition 29, and an evaporative surface 21 in fluid communication with the reservoir 28. The evaporative surface 21 may include additives (not shown) capable of converting electromagnetic energy into thermal energy.

Referring to FIGS. 8-11, a volatile composition dispenser 10 may include a housing 12 having a top portion 14 and a replaceable volatile composition container 20. It is contemplated that the volatile composition dispenser 10 may be configured for use in a variety of applications to deliver the volatile composition to the air and/or ultimately to a surface. The volatile composition dispenser 10 may be configured in various ways. For example, the volatile composition dispenser 10 may be configured as an electrical wall plug volatile composition dispenser (as shown in FIG. 12).

As stated previously, the volatile composition dispenser 10 may include a housing 12 and a replaceable volatile composition container 20. The housing 12 may include a top portion 14. In examples, the housing 12 may include a removable bottom portion 16.

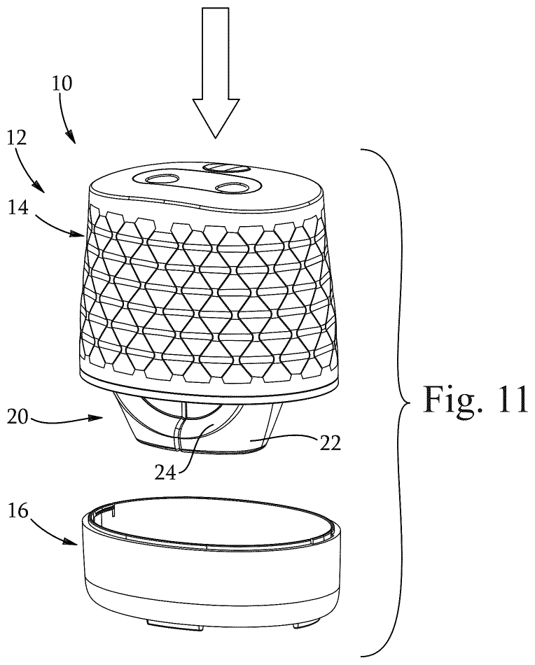

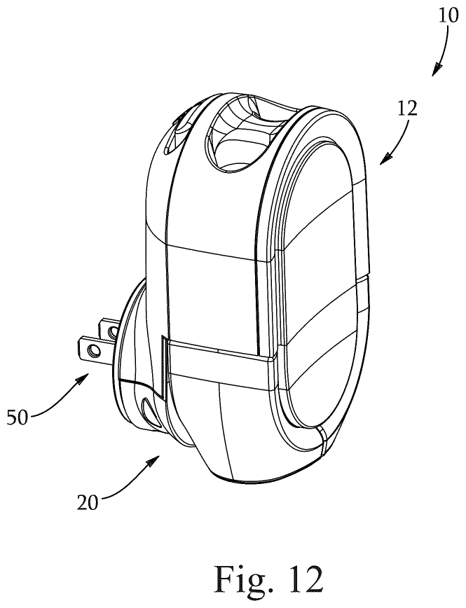

The replaceable volatile composition container 20 may releasably engage with the housing 12, and when the replaceable volatile composition container 20 is releasably engaged with the housing 12, the replaceable volatile composition container 20 may be positioned partially within the housing 12 (as depicted in FIGS. 11 and 12).

Referring now to FIG. 3, in examples, the sidewall 22 of the replaceable volatile composition container 20 at least partially encompasses a volatile composition reservoir 28. In examples, the volatile composition reservoir 28 may include a volatile composition 29. The sidewall 22 can be made of any suitable material. Suitable materials for construction of the sidewall include, but are not limited to, glass and plastic. The sidewall 22 may be constructed of any type of container that is suitable for holding volatile compositions.

The reservoir 28 may be configured to contain from about 5 milliliters (mL) to about 100 mL, alternatively from about 10 mL to about 50 mL, alternatively from about 15 mL to about 30 mL of fluid composition. The replaceable volatile composition container 20 may be configured to have multiple reservoirs 28, with each reservoir containing the same or different fluid composition and same or different fluid volumes. The reservoir 28 can be made of any suitable material for containing a fluid composition including glass, plastic, metal, or the like. The reservoir 28 may be transparent, translucent, or opaque or any combination thereof. For example, the reservoir 28 may be opaque with a transparent indicator of the level of fluid composition in the reservoir.

The sidewall 22 may form more than one volatile composition reservoir 28. In examples, the sidewall 22 may form one (not shown), two (as shown), three (not shown), four (not shown), or five (not shown) volatile composition reservoirs 28. It is also possible for each volatile composition reservoir 28 to have different types of volatile composition.

The term “volatile compositions” as used herein, refers to a material that comprises a vaporizable material. The term “volatile compositions,” thus includes (but is not limited to) compositions that are comprised entirely of a single volatile material. The terms “volatile materials,” “aroma,” “fragrance,” and “scents,” as used herein, include, but are not limited to pleasant or savory smells, and, thus, also encompass materials that function as insecticides, air fresheners, deodorants, aromacology, aromatherapy, or any other material that acts to condition, modify, or otherwise charge the atmosphere or to modify the environment. It should be understood that certain volatile compositions including, but not limited to perfumes, aromatic materials, and scented materials, will often comprise one or more volatile materials (which may form a unique and/or discrete unit comprised of a collection of volatile materials). It should be understood that the term “volatile composition” refers to compositions that have at least one volatile component, and it is not necessary for all of the component materials of the volatile composition to be volatile. The volatile compositions described herein may, thus, also have non-volatile components. It should also be understood that when the volatile compositions are described herein as being “emitted,” this refers to the volatilization of the volatile components thereof and does not require that the non-volatile components thereof be emitted. The volatile compositions of interest herein can be in any suitable form including, but not limited to, solids, liquids, gels, encapsulates, and combinations thereof.

The volatile composition may comprise from greater than 10 wt. %, alternatively greater than 20 wt. %, alternatively greater than 30 wt. %, alternatively greater than 40 wt. %, alternatively greater than 50 wt. %, alternatively greater than 60 wt. %, alternatively greater than 70 wt. %, alternatively greater than 85 wt. %, of perfume raw materials, based on the total weight of the volatile composition.

Referring still to FIG. 3, in examples, the volatile composition dispenser 10 may further include a delivery engine that may be used to transport the volatile composition to an evaporative surface 21. The delivery engine may include an evaporative surface 21. The evaporative surface 21 may be in fluid communication with the volatile composition reservoir 28. In such a configuration, the delivery engine may be placed next to one or more evaporative assistance elements, such as, but not limited to, a heater, light, or fan to volatilize the volatile composition into the air. The evaporative assistance elements may surround, or at least partially surround, the evaporative surface 21.

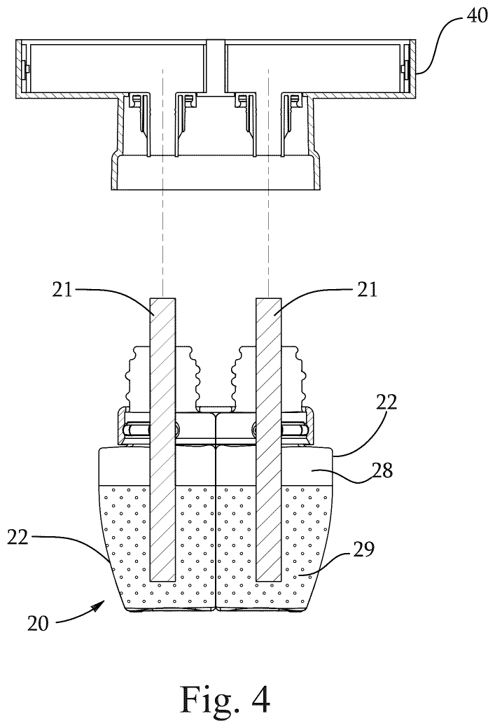

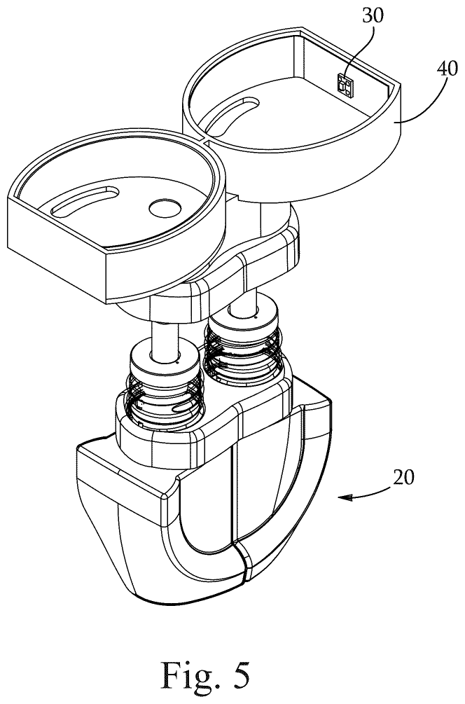

The evaporative assistance element may include an electromagnetic energy source 30. The electromagnetic energy source 30 may be positioned within an energy directing element 40. In examples, the energy directing element 40 may be positioned within the housing 12 (as shown in FIGS. 8-12). The electromagnetic energy source 30 may be positioned within the energy directing element 40 as shown in FIGS. 1-5, or may be positioned anywhere else along the boundary of the energy directing element 40. The energy directing element 40 may have a truncated elliptical shape as shown, or may have any other shape, such as a circle, a square, a triangle, an ovoid, or any other shape that allows for a electromagnetic energy source 30 to be placed in the energy directing element 40 and directed either directly or indirectly towards the evaporative surface 21. The electromagnetic energy may include photonic energy. Electromagnetic energy may be a superset of fields (electric or magnetic fields) and electromagnetic radiation. All electromagnetic radiation is photonic in nature, from radio waves to visible light to gamma rays. The photonic energy is chosen from the group consisting of: infrared light, visible light, and ultraviolet light, preferably infrared which includes near-infrared light and far-infrared light. The photonic energy may most preferably include near-infrared light.

The photonic energy may have a wavelength from about 10 nm to about 1 mm. Wavelengths from 10 nm (nanometers) to 1 mm (millimeters) encompasses ultraviolet, visible, and infrared portions of the spectrum. Preferably, the photonic energy may have a wavelength from about 700 nm to about 1400 nm. In examples, the photonic energy may have a wavelength of from 10 to 1,000,000 nm (where 1000000 nm equals 1 mm), from 10 to 500,000 nm, from 10 to 100,000 nm, from 10 to 50,000 nm, from 10 to 10,000 nm, from 10 to 5,000 nm, from 10 to 1,000 nm, from 10 to 500 nm, from 10 to 100 nm, from 100 to 1,000,000 nm, from 100 to 500,000 nm, from 100 to 100,000 nm, from 100 to 50,000 nm, from 100 to 10,000 nm, from 100 to 5,000 nm, from 100 to 1,000 nm, from 100 to 500 nm, from 500 to 1,000,000 nm, from 500 to 500,000 nm, from 500 to 100,000 nm, from 500 to 50,000 nm, from 500 to 10,000 nm, from 500 to 5,000 nm, from 500 to 1,400 nm, from 500 to 1,000 nm, from 700 to 1,000,000 nm, from 700 to 500,000 nm, from 700 to 100,000 nm, from 700 to 50,000 nm, from 700 to 10,000 nm, from 700 to 5,000 nm, from 700 to 1,400 nm, from 700 to 1,000 nm, from 1,000 to 1,000,000 nm, from 1,000 to 500,000 nm, from 1,000 to 100,000 nm, from 1,000 to 50,000 nm, from 1,000 to 10,000 nm, from 1,000 to 5,000 nm, from 1,000 to 1,400 nm, from 5,000 to 1,000,000 nm, from 5,000 to 500,000 nm, from 5,000 to 100,000 nm, from 5,000 to 50,000 nm, from 5,000 to 10,000 nm, from 10,000 to 1,000,000 nm, from 10,000 to 500,000 nm, from 10,000 to 100,000 nm, from 10,000 to 50,000 nm, from 50,000 to 1,000,000 nm, from 50,000 to 500,000 nm, from 50,000 to 100,000 nm, from 100,000 to 1,000,000 nm, from 100,000 to 500,000 nm, from 500,000 to 1,000,000 nm, or any values within the foregoing ranges or any ranges created thereby.

The electromagnetic energy source 30 may include a light source. The light source may include LEDs (Light Emitting Diode), such as infrared LEDs; lasers, such as VCSEL (Vertical-Cavity Surface-Emitting Laser), and EEL (Edge Emitting Laser); lamps, and combinations thereof. The vertical-cavity surface-emitting laser (VCSEL) is a type of semiconductor laser diode with laser beam emission perpendicular from the top surface, contrary to conventional edge-emitting semiconductor lasers (also called in-plane lasers) which emit from surfaces formed by cleaving the individual chip out of a wafer. EEL is an edge-emitting light-emitting diode is a type of LED structure where light emission occurs primarily along the edge of the semiconductor chip rather than from the surface.

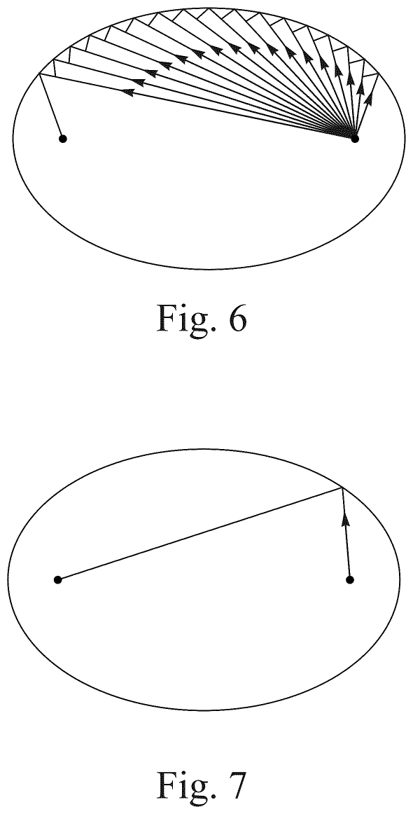

In examples, the electromagnetic energy source 30 may include a light source and the volatile composition dispenser 10 may further include a reflective surface 42 that reflects diffuse light towards the evaporative surface. The reflective surface 42 may be positioned on the energy directing element 40. The volatile composition dispenser 10 may include a reflective surface 42 that focuses the emitted radiation from the light source onto the evaporative surface 21. For this example, if the electromagnetic energy source 30 is on one side of the evaporative surface 21, the reflective surface 42 could be a concave structure on the other side of the evaporative surface 21 designed to ensure that all sides of the evaporative surface 21 receive the emitted radiation. A preferred example of the reflective surface 42 would be an ellipse-shaped reflective surface 42 (as shown) positioned on an inside surface of the energy directing element 40 where the evaporative surface 21 is placed at one focus of the ellipse formed by the energy directing element 40, and the electromagnetic energy source 30 is placed at the other focus. FIGS. 6 and 7 illustrate how the energy emitted by the electromagnetic energy source 30 at one focus of the ellipse, when reflected at the edges of the ellipse are mathematically deflected directly to the other focus of the ellipse where the evaporative surface 21 is positioned. It is a property of an ellipse that all rays emanating from one focus of the ellipse are reflected and concentrated on the opposite focus. In this example, a light source having a very wide dispersal angle may still have a substantial fraction of the light energy captured by the evaporating surface. Further, this arrangement ensures that the light is distributed around the (cylindrical) evaporating surface. This may be preferable to, for instance, a laser light source where the energy might be concentrated on a very small area of the evaporating surface. This might result in locally very high temperatures in a small area, while the majority of the evaporating surface remains unheated.

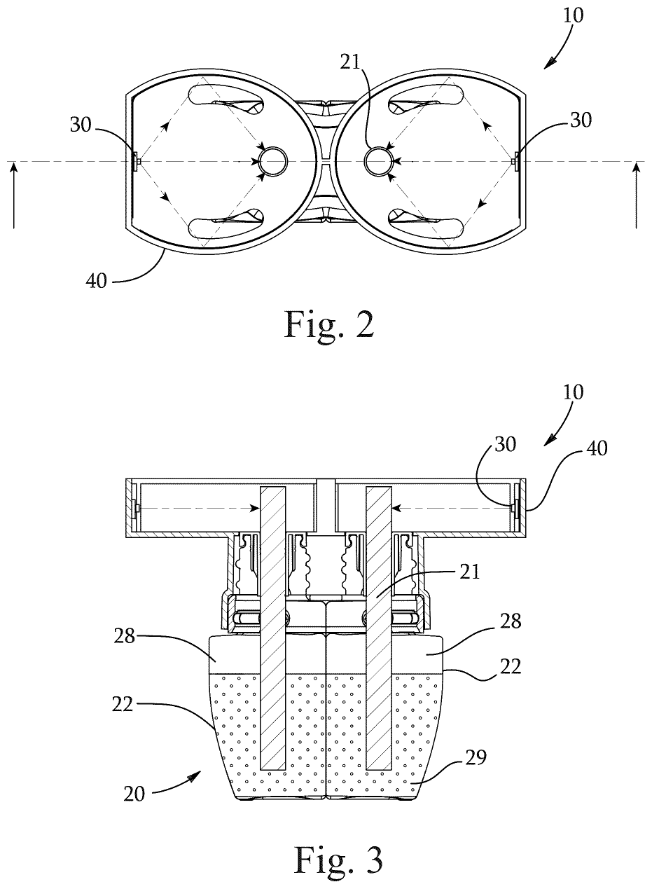

Referring to FIG. 2, due to the nature of the light source, it may be advantageous that the energy directing element 40 and the reflective surface 42 form a truncated ellipse, where the non-truncated portion directs the energy emitted from an energy source 30 located at a first focus of the truncated ellipse to a second focus of the truncated ellipse. The reflective property of the ellipse is retained while omitting a portion that does not contribute to directing the energy.

In examples, the light source may be positioned at various locations to the evaporative surface, including on the top, surrounding it, or inside the evaporative surface (not shown).

In examples, the light source can be controllable in on off duty cycle and energy output. This could control the energy used by the volatile composition dispenser. The duty cycle would be turning the electromagnetic energy source on and off, thereby controlling the evaporation rate of the volatile composition, and controlling the rate of release of fragrance as a result of varying the energy output of the electromagnetic energy source. US Patent Application Publications 2019/0216967 and 2014/0369895 are hereby incorporated by reference.

In examples, the light source may include at least two light sources that are independently controllable.

Referring now to FIG. 8, in examples, the evaporative assistance element may be electrically connected to an outlet connector 50. The outlet connector 50 may be electrically connected to the volatile composition dispenser 10 through a cord 52 through any means known in the art. In examples, the cord 52 may have a length of at least about 10 centimeters (cm), at least about 20 cm, at least about 30 cm, at least about 50 cm, from about 10 to about 300 cm, from about 10 to about 200 cm, from about 10 to about 150 cm, from about 10 to about 130 cm, from about 10 to about 100 cm, from about 10 to about 50 cm, from about 90 to about 300 cm, from about 90 to about 200 cm, from about 90 to about 150 cm, from about 90 to about 130 cm, from about 90 to about 100 cm, from about 100 to about 300 cm, from about 100 to about 200 cm, from about 100 to about 150 cm, from about 100 to about 130 cm, from about 130 to about 300 cm, from about 130 to about 200 cm, from about 130 to about 150 cm, from about 150 to about 300 cm, from about 150 to about 200 cm, or any values within the foregoing ranges or any ranges created thereby

The volatile composition dispenser 10 can be powered by a power source which may comprise an AC/DC outlet, a battery, or series of batteries, such as an AA battery, an AAA battery, a 9-volt battery, rechargeable battery, such as lithium/lithium ion batteries, and/or other suitable battery. In one embodiment, a solar power source, such as a solar cell, for example, can be used to power the device. In various embodiments, the solar cell (i.e., a photovoltaic cell) can be positioned on an outer portion of the dispenser or in communication with the device, such that the solar cell can receive light that can be transformed into energy to power the dispenser. Those of skill in the art, upon review of the present disclosure, will recognize that any other suitable method or device can be used to provide power to the dispenser.

In examples, instead of evaporating the volatile composition from an evaporative surface 21 of the delivery engine, the delivery engine may transport the volatile composition to a separate evaporative surface (not shown). The evaporative surface may be configured as a porous or semi-porous substrate, a bowl or plate, including a plastic, glass, or metal bowl or plate, and combinations thereof. The separate evaporative surface may include additives as described in this disclosure, either within (part of) the separate evaporative surface or on at least a portion of the surface area of the separate evaporative surface, as described in this disclosure.

The evaporative surface 21 may be configured in various ways. For example, the evaporative surface 21 may be in the form of a wick, membrane, gel, wax, porous or semi-porous substrate, including a felt pad. In a volatile composition dispenser 40 comprising more than one delivery engine associated with the same or different reservoirs (not shown), the delivery engines may be the same or may be different.

If the volatile composition dispenser utilizes a wick as the evaporative surface 21, the wick may be configured to have various different shapes and sizes. For example, the wick may have a cylindrical or an elongate cube shape or “D” shape or a partially or completely hollow shape. The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm. A wick may exhibit a density. The wick density may be in the range of about 0.100 grams/cm³ (“g/cc”) to about 1.0 g/cc.

A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as polyethylene (PE), high density polyethylene (HDPE), a polyethylene blend, or other polyolefins. The wick may include nonwovens, bi-component materials (such as a wick with a nonwoven overwrap that is impregnated with the photonic materials), synthetic sponges, ceramics, meshes, or combinations thereof. The wick may be at least partially hollow.

As previously stated, the evaporative surface may include additives capable of converting electromagnetic energy into thermal energy. In examples, the additives may be capable of increasing the absorptive capacity of the evaporative surface for photonic energy having a wavelength of from about 10 nm (nanometer) to about 1 mm (millimeter). In examples, the additives may be capable of increasing the absorptive capacity of the evaporative surface for photonic energy having a wavelength of from 10 to 1,000,000 nm (where 1000000 nm equals 1 mm), from 10 to 500,000 nm, from 10 to 100,000 nm, from 10 to 50,000 nm, from 10 to 10,000 nm, from 10 to 5,000 nm, from 10 to 1,000 nm, from 10 to 500 nm, from 10 to 100 nm, from 100 to 1,000,000 nm, from 100 to 500,000 nm, from 100 to 100,000 nm, from 100 to 50,000 nm, from 100 to 10,000 nm, from 100 to 5,000 nm, from 100 to 1,000 nm, from 100 to 500 nm, from 500 to 1,000,000 nm, from 500 to 500,000 nm, from 500 to 100,000 nm, from 500 to 50,000 nm, from 500 to 10,000 nm, from 500 to 5,000 nm, from 500 to 1,000 nm, from 1,000 to 1,000,000 nm, from 1,000 to 500,000 nm, from 1,000 to 100,000 nm, from 1,000 to 50,000 nm, from 1,000 to 10,000 nm, from 1,000 to 5,000 nm, from 5,000 to 1,000,000 nm, from 5,000 to 500,000 nm, from 5,000 to 100,000 nm, from 5,000 to 50,000 nm, from 5,000 to 10,000 nm, from 10,000 to 1,000,000 nm, from 10,000 to 500,000 nm, from 10,000 to 100,000 nm, from 10,000 to 50,000 nm, from 50,000 to 1,000,000 nm, from 50,000 to 500,000 nm, from 50,000 to 100,000 nm, from 100,000 to 1,000,000 nm, from 100,000 to 500,000 nm, from 500,000 to 1,000,000 nm, or any values within the foregoing ranges or any ranges created thereby.

The evaporative surface may have a spectral absorbance of from 10% to 100%, from 10% to 95%, from 10% to 90%, from 10% to 85%, from 10% to 80%, from 10% to 75%, from 10% to 70%, from 10% to 65%, from 10% to 60%, from 10% to 55%, from 10% to 50%, from 10% to 45%, from 10% to 40%, from 10% to 35%, from 10% to 30%, from 10% to 25%, from 10% to 20%, from 10% to 15%, from 15% to 100%, from 15% to 95%, from 15% to 90%, from 15% to 85%, from 15% to 80%, from 15% to 75%, from 15% to 70%, from 15% to 65%, from 15% to 60%, from 15% to 55%, from 15% to 50%, from 15% to 45%, from 15% to 40%, from 15% to 35%, from 15% to 30%, from 15% to 25%, from 15% to 20%, from 20% to 100%, from 20% to 95%, from 20% to 90%, from 20% to 85%, from 20% to 80%, from 20% to 75%, from 20% to 70%, from 20% to 65%, from 20% to 60%, from 20% to 55%, from 20% to 50%, from 20% to 45%, from 20% to 40%, from 20% to 35%, from 20% to 30%, from 20% to 25%, from 25% to 100%, from 25% to 95%, from 25% to 90%, from 25% to 85%, from 25% to 80%, from 25% to 75%, from 25% to 70%, from 25% to 65%, from 25% to 60%, from 25% to 55%, from 25% to 50%, from 25% to 45%, from 25% to 40%, from 25% to 35%, from 25% to 30%, from 30% to 100%, from 30% to 95%, from 30% to 90%, from 30% to 85%, from 30% to 80%, from 30% to 75%, from 30% to 70%, from 30% to 65%, from 30% to 60%, from 30% to 55%, from 30% to 50%, from 30% to 45%, from 30% to 40%, from 30% to 35%, from 35% to 100%, from 35% to 95%, from 35% to 90%, from 35% to 85%, from 35% to 80%, from 35% to 75%, from 35% to 70%, from 35% to 65%, from 35% to 60%, from 35% to 55%, from 35% to 50%, from 35% to 45%, from 35% to 40%, from 40% to 100%, from 40% to 95%, from 40% to 90%, from 40% to 85%, from 40% to 80%, from 40% to 75%, from 40% to 70%, from 40% to 65%, from 40% to 60%, from 40% to 55%, from 40% to 50%, from 40% to 45%, from 45% to 100%, from 45% to 95%, from 45% to 90%, from 45% to 85%, from 45% to 80%, from 45% to 75%, from 45% to 70%, from 45% to 65%, from 45% to 60%, from 45% to 55%, from 45% to 50%, from 50% to 100%, from 50% to 95%, from 50% to 90%, from 50% to 85%, from 50% to 80%, from 50% to 75%, from 50% to 70%, from 50% to 65%, from 50% to 60%, from 50% to 55%, from 55% to 100%, from 55% to 95%, from 55% to 90%, from 55% to 85%, from 55% to 80%, from 55% to 75%, from 55% to 70%, from 55% to 65%, from 55% to 60%, from 60% to 100%, from 60% to 95%, from 60% to 90%, from 60% to 85%, from 60% to 80%, from 60% to 75%, from 60% to 70%, from 60% to 65%, from 65% to 100%, from 65% to 95%, from 65% to 90%, from 65% to 85%, from 65% to 80%, from 65% to 75%, from 65% to 70%, from 70% to 100%, from 70% to 95%, from 70% to 90%, from 70% to 85%, from 70% to 80%, from 70% to 75%, from 75% to 100%, from 75% to 95%, from 75% to 90%, from 75% to 85%, from 75% to 80%, from 80% to 100%, from 80% to 95%, from 80% to 90%, from 80% to 85%, from 85% to 100%, from 85% to 95%, from 85% to 90%, from 90% to 100%, from 90% to 95%, from 95% to 100%, or any values within the foregoing ranges or any ranges created thereby. The term “spectral absorbance” refers to the ratio of absorbed radiant flux to incident radiant flux for a material at a prescribed wavelength or wavelengths. Radiant flux refers to the energy delivered per unit time (expressed in Watts) by a given input of electromagnetic radiation. Spectral absorbance may be measured using an instrument such as dispersive infrared spectrometer or a Fourier transform infrared (FTIR) spectrometer.

When additives are present on the surface of the evaporative surface, the additives may cover from 0.5% to 100%, from 0.5% to 95%, from 0.5% to 90%, from 0.5% to 85%, from 0.5% to 80%, from 0.5% to 75%, from 0.5% to 70%, from 0.5% to 65%, from 0.5% to 60%, from 0.5% to 55%, from 0.5% to 50%, from 0.5% to 45%, from 0.5% to 40%, from 0.5% to 35%, from 0.5% to 30%, from 0.5% to 25%, from 0.5% to 20%, from 0.5% to 15%, from 0.5% to 10%, from 0.5% to 5%, from 0.5% to 1%, from 1% to 100%, from 1% to 95%, from 1% to 90%, from 1% to 85%, from 1% to 80%, from 1% to 75%, from 1% to 70%, from 1% to 65%, from 1% to 60%, from 1% to 55%, from 1% to 50%, from 1% to 45%, from 1% to 40%, from 1% to 35%, from 1% to 30%, from 1% to 25%, from 1% to 20%, from 1% to 15%, from 1% to 10%, from 1% to 5%, from 5% to 100%, from 5% to 95%, from 5% to 90%, from 5% to 85%, from 5% to 80%, from 5% to 75%, from 5% to 70%, from 5% to 65%, from 5% to 60%, from 5% to 55%, from 5% to 50%, from 5% to 45%, from 5% to 40%, from 5% to 35%, from 5% to 30%, from 5% to 25%, from 5% to 20%, from 5% to 15%, from 5% to 10%, from 10% to 100%, from 10% to 95%, from 10% to 90%, from 10% to 85%, from 10% to 80%, from 10% to 75%, from 10% to 70%, from 10% to 65%, from 10% to 60%, from 10% to 55%, from 10% to 50%, from 10% to 45%, from 10% to 40%, from 10% to 35%, from 10% to 30%, from 10% to 25%, from 10% to 20%, from 10% to 15%, from 15% to 100%, from 15% to 95%, from 15% to 90%, from 15% to 85%, from 15% to 80%, from 15% to 75%, from 15% to 70%, from 15% to 65%, from 15% to 60%, from 15% to 55%, from 15% to 50%, from 15% to 45%, from 15% to 40%, from 15% to 35%, from 15% to 30%, from 15% to 25%, from 15% to 20%, from 20% to 100%, from 20% to 95%, from 20% to 90%, from 20% to 85%, from 20% to 80%, from 20% to 75%, from 20% to 70%, from 20% to 65%, from 20% to 60%, from 20% to 55%, from 20% to 50%, from 20% to 45%, from 20% to 40%, from 20% to 35%, from 20% to 30%, from 20% to 25%, from 25% to 100%, from 25% to 95%, from 25% to 90%, from 25% to 85%, from 25% to 80%, from 25% to 75%, from 25% to 70%, from 25% to 65%, from 25% to 60%, from 25% to 55%, from 25% to 50%, from 25% to 45%, from 25% to 40%, from 25% to 35%, from 25% to 30%, from 30% to 100%, from 30% to 95%, from 30% to 90%, from 30% to 85%, from 30% to 80%, from 30% to 75%, from 30% to 70%, from 30% to 65%, from 30% to 60%, from 30% to 55%, from 30% to 50%, from 30% to 45%, from 30% to 40%, from 30% to 35%, from 35% to 100%, from 35% to 95%, from 35% to 90%, from 35% to 85%, from 35% to 80%, from 35% to 75%, from 35% to 70%, from 35% to 65%, from 35% to 60%, from 35% to 55%, from 35% to 50%, from 35% to 45%, from 35% to 40%, from 40% to 100%, from 40% to 95%, from 40% to 90%, from 40% to 85%, from 40% to 80%, from 40% to 75%, from 40% to 70%, from 40% to 65%, from 40% to 60%, from 40% to 55%, from 40% to 50%, from 40% to 45%, from 45% to 100%, from 45% to 95%, from 45% to 90%, from 45% to 85%, from 45% to 80%, from 45% to 75%, from 45% to 70%, from 45% to 65%, from 45% to 60%, from 45% to 55%, from 45% to 50%, from 50% to 100%, from 50% to 95%, from 50% to 90%, from 50% to 85%, from 50% to 80%, from 50% to 75%, from 50% to 70%, from 50% to 65%, from 50% to 60%, from 50% to 55%, from 55% to 100%, from 55% to 95%, from 55% to 90%, from 55% to 85%, from 55% to 80%, from 55% to 75%, from 55% to 70%, from 55% to 65%, from 55% to 60%, from 60% to 100%, from 60% to 95%, from 60% to 90%, from 60% to 85%, from 60% to 80%, from 60% to 75%, from 60% to 70%, from 60% to 65%, from 65% to 100%, from 65% to 95%, from 65% to 90%, from 65% to 85%, from 65% to 80%, from 65% to 75%, from 65% to 70%, from 70% to 100%, from 70% to 95%, from 70% to 90%, from 70% to 85%, from 70% to 80%, from 70% to 75%, from 75% to 100%, from 75% to 95%, from 75% to 90%, from 75% to 85%, from 75% to 80%, from 80% to 100%, from 80% to 95%, from 80% to 90%, from 80% to 85%, from 85% to 100%, from 85% to 95%, from 85% to 90%, from 90% to 100%, from 90% to 95%, from 95% to 100%, or any values within the foregoing ranges or any ranges created thereby, of the exposed surface area of the evaporative surface. Surface here is defined as any surface where the electromagnetic energy is coming into contact with. For example, a wick with an inner core will have the electromagnetic energy directed into the inner core where it comes into contact with the additives. Additives are not strictly limited to only the surface. Additives may be internal to the wick surface where electromagnetic energy travels into the wick before encountering the adsorption additive.

When additives are present within the material making up the evaporative surface, the evaporative surface may include from 0.1 to 50 wt. %, from 0.1 to 40 wt. %, from 0.1 to 30 wt. %, from 0.1 to 20 wt. %, from 0.1 to 10 wt. %, from 0.1 to 5 wt. %, from 0.1 to 4 wt. %, from 0.1 to 3 wt. %, from 0.1 to 2 wt. %, from 0.1 to 1 wt. %, from 0.1 to 0.5 wt. %, from 0.5 to 50 wt. %, from 0.5 to 40 wt. %, from 0.5 to 30 wt. %, from 0.5 to 20 wt. %, from 0.5 to 10 wt. %, from 0.5 to 5 wt. %, from 0.5 to 4 wt. %, from 0.5 to 3 wt. %, from 0.5 to 2 wt. %, from 0.5 to 1 wt. %, from 1 to 50 wt. %, from 1 to 40 wt. %, from 1 to 30 wt. %, from 1 to 20 wt. %, from 1 to 10 wt. %, from 1 to 5 wt. %, from 1 to 4 wt. %, from 1 to 3 wt. %, from 1 to 2 wt. %, from 2 to 50 wt. %, from 2 to 40 wt. %, from 2 to 30 wt. %, from 2 to 20 wt. %, from 2 to 10 wt. %, from 2 to 5 wt. %, from 2 to 4 wt. %, from 2 to 3 wt. %, from 3 to 50 wt. %, from 3 to 40 wt. %, from 3 to 30 wt. %, from 3 to 20 wt. %, from 3 to 10 wt. %, from 3 to 5 wt. %, from 3 to 4 wt. %, from 4 to 50 wt. %, from 4 to 40 wt. %, from 4 to 30 wt. %, from 4 to 20 wt. %, from 4 to 10 wt. %, from 4 to 5 wt. %, from 5 to 50 wt. %, from 5 to 40 wt. %, from 5 to 30 wt. %, from 5 to 20 wt. %, from 5 to 10 wt. %, from 10 to 50 wt. %, from 10 to 40 wt. %, from 10 to 30 wt. %, from 10 to 20 wt. %, from 20 to 50 wt. %, from 20 to 40 wt. %, from 20 to 30 wt. %, from 30 to 50 wt. %, from 30 to 40 wt. %, from 40 to 50 wt. %, or any values within the foregoing ranges or any ranges created thereby, additives. Without being bound by theory, it is contemplated that when the additives are present within the material of the evaporative surface 21, the additives do not occupy any interstitial space of the evaporative surface 21 (for example, when the evaporative surface includes a wick). The additives may include organic or inorganic materials, or both. The inorganic materials may include but not limited to gold nanoparticles, black phosphorus, carbon nanomaterials, transition metal chalcogenides, transition metal chalcogenides, iron oxide nanoparticles, copper sulfide nanoparticles, or combinations thereof. Gold Nanoparticles (AuNPs): Gold nanoparticles are well-known for their strong surface plasmon resonance, which allows them to efficiently convert light, particularly in the near-infrared (NIR) region, into heat. They are extensively used in photothermal therapy due to their high thermal conversion efficiency and biocompatibility. Black Phosphorus: This material has gained attention for its excellent photothermal conversion efficiency and biodegradability. It absorbs light across a broad spectrum, including visible and NIR regions, making it suitable for biomedical applications. Carbon Nanomaterials: Carbon-based materials, such as graphene and carbon nanotubes, are known for their excellent light absorption and heat conversion properties. They are used in various photothermal applications due to their high thermal conductivity and stability. Transition Metal Chalcogenides: Materials such as molybdenum disulfide (MoS2) and tungsten disulfide (WS2) have been studied for their strong light absorption and efficient heat conversion. These materials are particularly effective in the NIR region. Iron Oxide Nanoparticles: These particles are used in magnetic hyperthermia and photothermal therapy. They have the ability to absorb NIR light and convert it into heat, which is useful for targeted cancer treatments. Copper Sulfide Nanoparticles: Known for their strong NIR absorption, copper sulfide nanoparticles are used in photothermal therapy and other applications requiring efficient light-to-heat conversion.

The organic materials may include but not limited to cyanines, diketopyrrolopyrroles (DPPs), croconaines, porphyrins, conductive polymers, heptamethine cyanines, or combinations thereof. Cyanines: These are a class of synthetic dyes with strong absorption in the near-infrared (NIR) region, making them effective for photothermal applications. They have been widely studied for their use in cancer therapy due to their ability to generate heat upon light absorption. Diketopyrrolopyrroles (DPPs): Known for their excellent photothermal conversion efficiency, DPPs are used in various applications, including PTT. They possess strong light absorption properties and are relatively easy to modify chemically. Croconaines: These dyes have been investigated for their photothermal properties and are known for their strong NIR absorption. They offer insights into the development of future photothermal agents, although some challenges regarding their bioavailability remain. Porphyrins: These naturally occurring compounds are effective in photothermal therapy due to their ability to absorb light and convert it into heat. They are often used in conjunction with other therapeutic modalities. Conductive Polymers: Polymers with conjugated structures, such as polyaniline and polypyrrole, have been shown to have high photothermal conversion efficiencies. They are valued for their stability and ease of synthesis. Heptamethine Cyanines: Compounds like IR825, IR780, and IR808 are noted for their superior photothermal efficiencies and photostability, making them promising candidates for clinical applications in photothermal therapy.

In examples, an evaporative surface with additives (treated evaporative surface) as described in this disclosure may have a faster or higher temperature rise/increase when compared to an evaporative surface without additives (untreated evaporative surface). In examples, the treated evaporative surface may have a temperature rise of 1° C. to 30° C. greater than a temperature rise of an untreated evaporative surface when exposed to the same amount of electromagnetic energy. In examples, the treated evaporative surface may have a temperature rise of from 1° C. to 30° C., from 1° C. to 20° C., from 1° C. to 12° C., from 1° C. to 10° C., from 1° C. to 9° C., from 1° C. to 8° C., from 1° C. to 7° C., from 1° C. to 6° C., from 1° C. to 3° C., from 3° C. to 30° C., from 3° C. to 20° C., from 3° C. to 12° C., from 3° C. to 10° C., from 3° C. to 9° C., from 3° C. to 8° C., from 3° C. to 7° C., from 3° C. to 6° C., from 6° C. to 30° C., from 6° C. to 20° C., from 6° C. to 12° C., from 6° C. to 10° C., from 6° C. to 9° C., from 6° C. to 8° C., from 6° C. to 7° C., from 7° C. to 30° C., from 7° C. to 20° C., from 7° C. to 12° C., from 7° C. to 10° C., from 7° C. to 9° C., from 7° C. to 8° C., from 8° C. to 30° C., from 8° C. to 20° C., from 8° C. to 12° C., from 8° C. to 10° C., from 8° C. to 9° C., from 9° C. to 30° C., from 9° C. to 20° C., from 9° C. to 12° C., from 9° C. to 10° C., or any values within the foregoing ranges or any ranges created thereby, greater than a temperature rise of an untreated evaporative surface when exposed to the same amount of electromagnetic energy.

### EXAMPLES

In a proof-of-concept experiment, a polymer having good absorption in the near infrared (NIR) spectrum was introduced to an existing sintered polypropylene wick. Temperature measurement of an untreated wick vs. a treated wick showed that the wick can be made to efficiently absorb infrared radiation.

Temperature measurement of the wick can be done using a fine wire thermocouple which is embedded in the wick material by removing a small portion of the wick to allow the thermocouple to be placed along the surface of the wick, and the temperature measuring portion of the thermocouple bonded to the wick material using a small bead of epoxy.

Depositing of the polymer can be done by creating a solution of polymer, polyaniline (emeraldine salt), in 1-methyl-2-pyrrolidinone solvent. Heating the solvent to approximately 60° C. and stirring on a stir plate increases the solubility of the polymer. The wick is then immersed in the polymer solution, and the porous material becomes saturated with the solution. The wick can then be dried, leaving only the polymer deposited in the matrix of the wick.

Evaluation of the NIR absorbance of the wick was evaluated by placing a NIR emitting light emitting diode (LED, having a wavelength of 850 nm) in proximity to the wick, with the temperature sensing portion of the thermocouple aligned with the optical center of the NIR LED. An untreated wick had a temperature rise of 1.2° C. while the treated wick had a temperature rise of 9.3° C.

Another proof-of-concept experiment uses the graphene coating material as absorbing agent. The wick is partially dipped into the graphene coating material in a liquid form, then dried out at the room temperature. Absorbing agents such as graphene has reported an exceptionally large thermal conductivity up to 5300 W·m⁻¹·K⁻¹.

Note that this technique has the limitation that the light absorbing additive was added to the finished wick, and thus was deposited on the surfaces of the constituent plastic particles of the sintered matrix. In the proposed application, the additive would be introduced in the base material and thus distributed uniformly within the constituent particles. Nevertheless, a similar result would be expected.

This proof-of-concept shows that the introduction of light absorbing additives can result in the absorption of light energy, raising the temperature of the wick and thus enhancing evaporation of volatile compositions.

### Combinations

A. A volatile composition dispenser comprising:
a reservoir comprising a volatile composition;
an evaporative surface in fluid communication with the reservoir; and
an electromagnetic energy source, wherein:
the evaporative surface comprises additives capable of converting electromagnetic energy into thermal energy.

B. The volatile composition dispenser as disclosed in A, wherein the reservoir is positioned within a replaceable volatile composition container.

C. The volatile composition dispenser as disclosed in B, wherein the volatile composition dispenser comprises a housing and the replaceable volatile composition container is releasably engaged with the housing.

D. The volatile composition dispenser as disclosed in A, wherein the electromagnetic energy comprises photonic energy.

E. The volatile composition dispenser of as disclosed in D, wherein the photonic energy is chosen from the group consisting of: infrared, visible, and ultraviolet light.

F. The volatile composition dispenser of as disclosed in D, wherein the photonic energy has a wavelength from about 10nm to about 1 mm.

G. The volatile composition dispenser as disclosed in A, wherein the electromagnetic energy source comprises a light source.

H. The volatile composition dispenser of as disclosed in G, wherein the light source is chosen from the group consisting of LEDs (Light Emitting Diode) and lasers.

I. The volatile composition dispenser of as disclosed in G, wherein the electromagnetic energy source comprises a light source and the volatile composition dispenser further comprises a reflective surface that reflects diffuse light towards the evaporative surface.

J. The volatile composition dispenser of as disclosed in G, wherein the light source is chosen from the group consisting of: infrared LED (Light Emitting Diode), VCSEL (Vertical-Cavity Surface-Emitting Laser), and EEL (Edge Emitting Laser).

K. The volatile composition dispenser of as disclosed in G, wherein the light source is positioned at various locations to the wick, including on the top, surrounding it, or inside the evaporative surface.

L. The volatile composition dispenser of as disclosed in G, wherein the light source can be controllable in on off duty cycle and energy output.

M. The volatile composition dispenser of as disclosed in G, wherein the light source can have more than one light source independently controllable.

N. The volatile composition dispenser as disclosed in A, wherein the additives are capable of increasing the absorptive capacity of the evaporative surface for photonic energy having a wavelength of from about 10 nm to about 1 mm.

O. The volatile composition dispenser as disclosed in A, wherein the evaporative surface has spectral absorbance of from about 10% to 100%.

P. The volatile composition dispenser as disclosed in A, wherein the additive comprises inorganic materials.

Q. The volatile composition dispenser as disclosed in P, wherein the inorganic materials are chosen from the group consisting of: gold nanoparticles, black phosphorus, carbon nanomaterials, transition metal chalcogenides, transition metal chalcogenides, iron oxide nanoparticles, and copper sulfide nanoparticles.

R. The volatile composition dispenser as disclosed in A, wherein the additive comprises organic materials.

S. The volatile composition dispenser as disclosed in R, wherein the organic materials are chosen from the group consisting of: cyanines, diketopyrrolopyrroles (DPPs), croconaines, porphyrins, conductive polymers, and heptamethine cyanines.

T. The volatile composition dispenser as disclosed in A, wherein the evaporative surface comprises a mix of different additives with different adsorption wavelengths.

U. The volatile composition dispenser of claim 20, wherein the additives both absorb light and can function as fluid transport.

V. The volatile composition dispenser as disclosed in A, wherein the additive is embedded in the evaporative surface.

W. The volatile composition dispenser as disclosed in A, wherein the additive is deposited onto at least a portion of the evaporative surface.

X. The volatile composition dispenser as disclosed in A, wherein the evaporative surface is chosen from the group consisting of a wick, a membrane, gel, wax, a porous substrate, and a semi-porous substrate.

Y. The volatile composition dispenser as disclosed in A, a method of operating the volatile composition dispenser as disclosed in A.

Z. A replaceable volatile composition container comprising:
a reservoir comprising a volatile composition; and
an evaporative surface in fluid communication with the reservoir, wherein:
the evaporative surface comprises additives capable of converting electromagnetic energy into thermal energy.

AA. The replaceable volatile composition container as disclosed in Z, wherein a light source is positioned inside the evaporative surface.

BB. The replaceable volatile composition container as disclosed in AA, wherein the light source is chosen from the group consisting of LEDs (Light Emitting Diode) and lasers.

CC. The replaceable volatile composition container as disclosed in AA, wherein the light source is chosen from the group consisting of: infrared LED (Light Emitting Diode), VCSEL (Vertical-Cavity Surface-Emitting Laser), and EEL (Edge Emitting Laser).

DD. The replaceable volatile composition container as disclosed in AA, wherein the light source can be controllable in on off duty cycle and energy output.

EE. The replaceable volatile composition container as disclosed in AA, wherein the light source can have more than one light source independently controllable.

FF. The replaceable volatile composition container as disclosed in Z, wherein the additives are capable of increasing the absorptive capacity of the evaporative surface for photonic energy having a wavelength of from about 10 nm to about 1 mm.

GG. The replaceable volatile composition container as disclosed in Z, wherein the evaporative surface has spectral absorbance of from about 10% to 100%.

HH. The replaceable volatile composition container as disclosed in Z, wherein the additive comprises inorganic materials.

II. The replaceable volatile composition container as disclosed in HH, wherein the inorganic materials are chosen from the group consisting of: gold nanoparticles, black phosphorus, carbon nanomaterials, transition metal chalcogenides, transition metal chalcogenides, iron oxide nanoparticles, and copper sulfide nanoparticles.

JJ. The replaceable volatile composition container as disclosed in Z, wherein the additive comprises organic materials.

KK. The replaceable volatile composition container as disclosed in JJ, wherein the organic materials are chosen from the group consisting of: cyanines, diketopyrrolopyrroles (DPPs), croconaines, porphyrins, conductive polymers, and heptamethine cyanines.

LL. The replaceable volatile composition container as disclosed in Z, wherein the evaporative surface comprises a mix of different additives with different adsorption wavelengths.

MM. The replaceable volatile composition container as disclosed in LL, wherein the additives both absorb light and can function as fluid transport.

NN. The replaceable volatile composition container as disclosed in Z, wherein the additive is embedded in the evaporative surface.

OO. The replaceable volatile composition container as disclosed in Z, wherein the additive is deposited onto at least a portion of the evaporative surface.

PP. The replaceable volatile composition container as disclosed in Z, wherein the evaporative surface is chosen from the group consisting of a wick, a membrane, gel, wax, a porous substrate, and a semi-porous substrate.

QQ. The volatile composition dispenser as disclosed in A, wherein the electromagnetic ranges from 0.1 watts to 10 watts.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the claims that follow, the articles “a”, “an”, and “the” include singular and plural references unless the context clearly dictates otherwise. As such, the terms “a” or “an”, “one or more” and “at least one” can be used interchangeably herein. Thus, for example, “a component” may include one or more components unless the reference is specifically indicated as being singular.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A volatile composition dispenser comprising:
a reservoir comprising a volatile composition;
an evaporative surface in fluid communication with the reservoir; and
an electromagnetic energy source,
wherein the evaporative surface comprises additives capable of converting electromagnetic energy into thermal energy.

2. The volatile composition dispenser of claim 1, wherein the reservoir is positioned within a replaceable volatile composition container.

3. The volatile composition dispenser of claim 2, wherein the volatile composition dispenser comprises a housing, and the replaceable volatile composition container is releasably engaged with the housing.

4. The volatile composition dispenser of claim 1, wherein the electromagnetic energy comprises photonic energy selected from the group consisting of: infrared, visible, and ultraviolet light.

5. The volatile composition dispenser of claim 4, wherein the photonic energy has a wavelength from about 10nm to about 1mm.

6. The volatile composition dispenser of claim 1, wherein the electromagnetic energy source comprises a light source and the volatile composition dispenser further comprises a reflective surface that reflects diffuse light towards the evaporative surface.

7. The volatile composition dispenser of claim 1, wherein the electromagnetic energy source comprises a light source, wherein the light source is chosen from the group consisting of LED (Light Emitting Diode), laser, and a combination thereof.

8. The volatile composition dispenser of claim 7, wherein the light source is chosen from the group consisting of: infrared LED (Light Emitting Diode), VCSEL (Vertical-Cavity Surface-Emitting Laser), and EEL (Edge Emitting Laser).

9. The volatile composition dispenser of claim 7, wherein the light source is positioned at various locations to the evaporative surface, including on the top, surrounding it, or inside the evaporative surface.

10. The volatile composition dispenser of claim 7, wherein the light source can be controllable in on off duty cycle and energy output.

11. The volatile composition dispenser of claim 7, wherein the light source comprises more than one independently controllable light source.

12. The volatile composition dispenser of claim 1, wherein the additives are capable of increasing the absorptive capacity of the evaporative surface for photonic energy having a wavelength of from about 10nm to about 1mm.

13. The volatile composition dispenser of claim 1, wherein the evaporative surface has spectral absorbance of from about 10% to 100%.

14. The volatile composition dispenser of claim 1, wherein the additive comprises inorganic materials selected from the group consisting of: gold nanoparticles, black phosphorus, carbon nanomaterials, transition metal chalcogenides, transition metal chalcogenides, iron oxide nanoparticles, copper sulfide nanoparticles, and combinations thereof.

15. The volatile composition dispenser of claim 1, wherein the additive comprises organic materials selected from the group consisting of: cyanines, diketopyrrolopyrroles (DPPs), croconaines, porphyrins, conductive polymers, heptamethine cyanines, and combinations thereof.

16. The volatile composition dispenser of claim 1, wherein the evaporative surface comprises a mix of different additives with different adsorption wavelengths, wherein the different additives both absorb light and can function as fluid transport.

17. The volatile composition dispenser of claim 1, wherein the additive is embedded in the evaporative surface.

18. The volatile composition dispenser of claim 1, wherein the additive is deposited onto at least a portion of the evaporative surface.

19. The volatile composition dispenser of claim 1, wherein the evaporative surface is chosen from the group consisting of a wick, a membrane, gel, wax, a porous substrate, and a semi-porous substrate.

20. A replaceable volatile composition container comprising:
a reservoir comprising a volatile composition; and
an evaporative surface in fluid communication with the reservoir,
wherein the evaporative surface comprises additives capable of converting electromagnetic energy into thermal energy, and
optionally wherein a light source is positioned inside the evaporative surface.
